(19) United States
(12) Patent Application Publication
(10) Pub. No.: US 2025/0011819 A1
(43) Pub. Date: Jan. 9, 2025

(54) METHOD FOR PRODUCING PRODUCT GAS COMPRISING METHANE

(71) Applicant: Nature Energy Biogas A/S, Odense SØ (DK)

(72) Inventors: Martin Dan JEPPESEN, Odense V (DK); Brian Dahl JØNSON, Odense SØ (DK); Lina Johanna Nåbo ANDERSEN, Morud (DK)

(73) Assignee: Nature Energy Biogas A/S, Odense SØ (DK)

(21) Appl. No.: 18/713,600

(22) PCT Filed: Nov. 29, 2022
(86) PCT No.: PCT/DK2022/050251

§ 371 Date: May 24, 2024

(30) Foreign Application Priority Data
Nov. 29, 2021 (WO) PCT/DK2021/050348

(51) Int. Cl. C12P 5/02; C12M 1/107; C12M 1/42; C12P 3/00

(52) U.S. Cl. CPC C12P 5/023; C12M 21/04; C12M 35/02; C12M 35/04; C12P 3/00

(57) ABSTRACT

A method for producing product gas (PG) comprising methane is disclosed, the method comprising the steps of providing a biomass (BM), subjecting the biomass (BM) to an anaerobic digestion to produce biogas (BG) and a biomass digestate (BMD), separating the biomass digestate (BMD) into a liquid digestate fraction (LDF) and a solid digestate fraction (SDF), and subjecting the liquid digestate fraction to microbial electrolysis cell (MEC) processing to produce methane and/or hydrogen gas. Also, a system for producing product gas (PG) comprising methane is disclosed.

### FIELD OF INVENTION

The invention relates to a method for producing product gas comprising methane, optionally also comprising hydrogen gas. Particularly, the invention relates to a method for producing product gas comprising methane according to the claims.

### BACKGROUND

Production of biogas from various biomass has become increasingly popular over the years. Biogas represents both an extra fuel gas and also a greener alternative to fossil gas and other fossil fuels.

Therefore, a higher production of biogas is desirable, and much effort has been put into increasing the yield of biogas. For example, two step anaerobic digestion has shown to provide favorable biogas yield, as shown in WO 2020/099651 A1.

Also, there has been focus on increasing the possible usable biomass fractions, e.g. by increasing cultivation of suitable energy crops and/or by treating previously unusable fractions to become processable for biogas production.

Nevertheless, a need for increasing the biogas output still exists.

Also, obtaining valuable biproducts from the biogas production has gained increasing focus.

At the same time, other gasses e.g. for use as fuels are also becoming increasingly high in demand, e.g. hydrogen gas.

It is an object of the invention to solve the above problems and challenges.

### SUMMARY

The invention relates to a method for producing product gas comprising methane, the method comprising the steps of
providing a biomass,
subjecting the biomass to an anaerobic digestion to produce biogas and a biomass digestate,
separating the biomass digestate into a liquid digestate fraction and a solid digestate fraction, and
subjecting the liquid digestate fraction to microbial electrolysis cell processing to produce methane and/or hydrogen gas.

One advantage of the invention may be that an improvement of the product gas production may be obtained. Depending on specific microbial electrolysis cell employed, the further product gas may comprise additional biogas and/or hydrogen gas. By utilizing the biomass digestate, which may typically represent a low value or even negative fraction such as a fertilizer fraction to be spread on fields, additional amounts of fuel gas may be extracted.

In an embodiment of the invention, it should be understood that the microbial electrolysis cell may produce methane gas or may produce a combination of methane and hydrogen gas, depending on its configuration, in particular if a so-called membrane configuration is employed or not. The produced methane gas or combination of methane gas and hydrogen gas may thus be referred to as product gas comprising methane gas or a combination of methane gas and hydrogen gas. Hence, the liquid digestate fraction is subjected to microbial electrolysis cell processing to produce product gas comprising methane gas or a combination of methane gas and hydrogen gas.

In the present context, the term “product gas” may refer to gas comprising methane gas and optionally also hydrogen gas. Biogas comprising methane is produced from anaerobic digestion. This biogas may be referred to as AD product gas. Also, methane gas and/or hydrogen gas is produced from the microbial electrolysis cell processing. This gas may be referred to as MEC product gas. In embodiments where the MEC is provided in a two-chamber configuration to produce methane gas and hydrogen gas, the MEC product gas may typically be extracted in two separate gas fractions, namely a first MEC product gas comprising methane gas and a second MEC product gas comprising hydrogen gas.

Additionally, such increased product gas production may be obtained at in a cost-effective way, since the operation of the microbial electrolysis cell is optimized to both ensure organic matter for conversion in the microbial electrolysis cell while at the same time avoiding or minimizing production breakdowns and frequent maintenance outages related to clogging of the microbial electrolysis cell. Typically, biomass digestate has a content of unconverted components, such lignocellulosic biomass components including straw components etc. Therefore, these are typically considered unfavorable for processing in microbial electrolysis cells, and instead focus may typically be directed towards optimizing the anaerobic digestion reaction, e.g. by performing multi-step anaerobic digestion. However, such measures may increase costs to an unfavorable level, even if a more efficient biogas extraction is possible from a technical point of view.

A further advantage of the invention may be that hydrogen gas may be produced in a relatively effective manner. Particularly, by using microbial electrolysis cell processing rather than conventional electrolysis, the voltage applied for hydrogen gas generation may be lowered. In this respect it is noted that the voltage may be reduced both below the typical optimum voltage levels of conventional electrolysis facilities, and in many cases even below the theoretical minimum voltage levels necessary for conventional hydrogen electrolysis.

Yet a further advantage of the invention may be that by using a different composition of microorganisms, particularly comprising a higher relative content electrogenic microorganisms, the breakdown of the remaining biomass may be more efficient than further anaerobic processing based on conventional microorganism cultures.

As used herein, the term “biomass” is intended to mean material of organic origin. The biomass may also be described as soft biomass comprising cellulosic and herbaceous types of biomass, such as wheat straw, corn stover, rice straw, grass, and bagasse. In an embodiment of the invention, the biomass comprises animal feces, such as livestock feces. Such animal feces may be provided in the form of manure and/or deep litter. Additionally, the biomass may comprise solid and/or liquid or pumpable fractions. Solid fractions may e.g. include plant pulp (such as potato pulp), grass, etc. Liquid and pumpable fractions may e.g. include industrial waste, such as food waste. Finally, the biomass may also comprise biomass fractions containing high content of energy (e.g. having a high COI) value). Such fractions may include molasses, fats, etc.

In an embodiment of the invention, the biomass may comprise lignin and cellulose as main constituents for degrading into biogas.

As used herein, the term “biogas” is intended to mean a product gas comprising methane gas. Biogas is obtained from degradation of biological material, such as biomass. Typically, raw or unprocessed biogas comprises methane gas and carbon dioxide gas as its main constituents, whereas processed biogas is composed of methane or at least mainly of methane. Minor amounts of e.g. hydrogen sulfide and water vapor may also be present. Typically, the raw biogas may be upgraded or purified to increase the relative content of methane, e.g. in view of legal limits on minimum methane content and/or maximum content of certain other gasses. The content of other constituents in processed biogas may vary, e.g. due to legal requirements and also based on the specific composition of the used biomass. In some embodiments, raw or unprocessed biogas comprises methane in an amount of at least 40% by volume of the biogas, such as 40-90% by volume of the biogas. In some embodiments, the processed biogas comprises methane in an amount of at least 95% by volume of the biogas, such as 95-99.99% by volume of the biogas.

As used herein, the term “anaerobic digestion” refers to the breakdown of biomass by microbes at oxygen deficient conditions, i.e. at zero or very low content of oxygen. In the present context, microorganisms facilitate the breakdown of biomass, eventually resulting in biogas containing methane and carbon dioxide as its main constituents. Such microorganisms may e.g. comprise hydrolysis performing microorganisms, acidogenic microorganisms, acetogenic microorganisms, methanogenic microorganisms etc. It is noted that the terms “anaerobic digestion reactor”, “anaerobic digester”, and “anaerobic reactor” may be used interchangeably.

As used herein, the term “biomass digestate” refers to remaining material after anaerobic digestion of biomass, i.e. when biogas has been collected. Biomass digestate can be fibrous and contain structural plant matter including lignin and cellulose. The biomass digestate may also contain minerals and remnants of bacteria.

As used herein, the term “output digestate” refers to remaining material after microbial electrolysis cell processing of the biomass digestate. Thus, the output digestate may also be referred to as “MEC processed digestate” or simply “processed digestate”.

As used herein, the term “liquid digestate fraction” is intended to mean the fraction having the lowest dry matter after a separation step of the biomass digestate. The amount of suspended solids may e.g. be around 2-6% by weight, such as 4% by weight, but typically varies from 0-10% by weight of the liquid digestate fraction.

As used herein, the term “solid digestate fraction” is intended to mean the fraction having the highest dry matter after a separation step. The amount of suspended solids may e.g. be around 20-25% by weight, but may vary from 10-95% by weight of the solid digestate fraction.

As used herein, the term “microbial electrolysis cell” is used in the context of processing by electrogenic microorganisms consuming a biomass and its degradation products to produce methane and/or hydrogen gas when an external voltage is applied. More specific, the terms “microbial electrolysis cell” and “microbial electrolysis cell reactor” is used interchangeably to refer to the device configured for performing such process.

As used herein, the term “dry matter” is intended to mean the residual when water is evaporated.

As used herein, the term volatile solids (VS) shall mean the organic part of dry matter. Usually this is measured by heating a sample (which has been dried at 105 degrees Celsius) to 450 degrees Celsius, so that only salts and ashes remain.

According to an advantageous embodiment of the invention, the anaerobic digestion comprises a first anaerobic digestion step and a second anaerobic digestion step.

One advantage of the above embodiment may be that the yield of methane may be increased in the anaerobic digestion.

In an embodiment of the invention, the first anaerobic digestion step is performed in a first anaerobic digestion reactor, and the second anaerobic digestion step is performed in a second anaerobic digestion reactor.

According to an advantageous embodiment of the invention, the processing time of the first anaerobic digestion step exceeds the processing time of the second anaerobic digestion step.

In an embodiment of the invention, the processing time of the first anaerobic digestion step exceeds the processing time of the second anaerobic digestion step by at least 20%, such as at least 30%, such as at least 40%.

According to an advantageous embodiment of the invention, the anaerobic digestion comprises mixing.

An advantage of the above embodiment may be that a more efficient anaerobic digestions may be obtained, e.g. by avoiding floating layers and also by keeping a uniform distribution of methanogenic bacteria and undigested biomass components.

According to an advantageous embodiment of the invention, anaerobic digestion is performed in at least one anaerobic digestion reactor comprising at least one continuously stirred-tank reactor.

In an embodiment of the invention, the at least one anaerobic digestion reactor comprises a first anaerobic digestion reactor being a continuously stirred-tank reactor and a second anaerobic digestion reactor being a separate continuously stirred-tank reactor.

According to an advantageous embodiment of the invention, anaerobic digestion is performed in at least one anaerobic digestion reactor.

In some embodiments two or more anaerobic digestion reactors may be used in parallel, e.g. to obtain the desired scale at a given processing site.

In some embodiments two or more anaerobic digestion reactors may be used in series, e.g. two subsequent anaerobic digestion reactors, which may provide for an improved biogas production.

According to an advantageous embodiment of the invention, the anaerobic digestion reactor is a continuous anaerobic digestion reactor.

Thus, in the above embodiment, the biomass is continuously fed to the least one anaerobic digestion reactor.

According to an advantageous embodiment of the invention, the biomass is continuously fed to a solid-liquid separation arrangement for separating the biomass digestate into a liquid digestate fraction and a solid digestate fraction.

According to an advantageous embodiment of the invention, the product gas consists of biogas.

According to an advantageous embodiment of the invention, the product gas consists of biogas and optionally hydrogen gas.

According to an advantageous embodiment of the invention, the product gas further comprises hydrogen gas produced in the microbial electrolysis cell.

According to an advantageous embodiment of the invention, the method further comprises collection of product gas.

It is noted that the collection of the produced product gas may in principle be continuous or during distinct time periods. Also, it is noted that the collection of product gas may comprise individually collecting gas from anaerobic digestion and the microbial electrolysis cell processing. The collected gas from the anaerobic digestion may be kept separate from the gas collected from the microbial electrolysis cell processing, or the two gas fractions may be mixed.

According to an advantageous embodiment of the invention, the method comprises collecting product gas comprising methane from the at least one anaerobic digestion reactor and separately collecting methane gas and/or hydrogen gas from the microbial electrolysis cell.

Thus, in the above embodiment, the anaerobic digestion is performed in at least one anaerobic digestion reactor. Also, as noted above, the collection of product gas may be continuous, or only at distinct time periods. As described elsewhere, the product gas collected from the microbial electrolysis cell may comprise methane and/or hydrogen gas.

According to an advantageous embodiment of the invention, the method further comprises a step of subjecting the biomass or a digestate and/or fraction thereof to a cavitation treatment.

An advantage of the above embodiment may be that the biomass is made more susceptible to processing by anaerobic digestion and/or by microbial electrolysis cell processing. In more detail, cavitation may physically break down fibrous biomass into much smaller particles, whereby the relative surface area of the particles, and thus the susceptibility to anerobic digestion and/or by microbial electrolysis cell processing, may be significantly increased.

According to an advantageous embodiment of the invention, the method further comprises a step of subjecting the biomass to a cavitation treatment before anaerobic digestion.

According to an advantageous embodiment of the invention, the cavitation treatment comprises treating the biomass during the anaerobic digestion.

In an embodiment of the invention, the cavitation treatment is performed external to the anaerobic digestion reactor. As an example embodiment, the biomass is continuously fed from the anaerobic digestion reactor to be processed by cavitation and thereafter being fed back into the anaerobic digestion reactor.

According to an advantageous embodiment of the invention, the cavitation treatment comprises treating the biomass digestate before the separation

An advantage of the above embodiment may be that the cavitation treatment may reduce the need for solid-liquid separation, and thereby provide a more cost-efficient process, where the need for maintenance of filters and other separation equipment may be reduced.

According to an advantageous embodiment of the invention, the cavitation treatment comprises treating the liquid digestate fraction before the microbial electrolysis cell processing.

According to an advantageous embodiment of the invention, the cavitation treatment comprises ultrasonic cavitation treatment.

In an embodiment of the invention, the method further comprises pretreating the biomass before the anaerobic digestion.

According to an advantageous embodiment of the invention, the method further comprises a step of pretreating, the step of pretreating comprising subjecting the biomass to a pressure below 2 bar and a temperature in the range of 65 to 100 degrees Celsius.

In an embodiment of the invention the step of pretreating comprises subjecting the biomass to a pressure in the range of 0.5 to 2 bar.

According to an embodiment of the invention, the step of pretreating is performed for 2 hours or less, such as 1 hour or less, such as 45 minutes or less, such as 30 minutes or less, or 15 minutes or less. According to an embodiment of the invention, the pretreatment has a duration of in the range of 5 minutes to 2 hours, such as in the range of 5 minutes to 1 hour.

According to an embodiment of the invention, the pH in the step of pretreating is in the range of 2 to 11, such as in the range of 2 to 9, such as in the range of 3 to 7, such as in the range of 3 to 4.

In an embodiment of the invention, the method further comprises posttreating the biomass digestate before the separation step.

According to an advantageous embodiment of the invention, the method further comprises posttreating the biomass digestate before the separation step, where the posttreating comprises subjecting the biomass digestate to a temperature above 150 degrees Celsius.

In an embodiment of the invention, the method further comprises posttreating the biomass digestate before the separation step, where the posttreating comprises subjecting the biomass digestate to a temperature in the range of 150 to 230 degrees Celsius, such as in the range of 170 to 210 degrees Celsius, such as in the range of 180 to 200 degrees Celsius.

In an embodiment of the invention, the posttreatment step is performed for no more than 1 hour, such as no more than 45 minutes, such as no more than 30 minutes, such as in the range of 10 to 30 minutes.

In an embodiment of the invention, the pH in the posttreatment step is in the range of 2 to 10.

In an embodiment of the invention, the pressure in the posttreatment step is in the range of 5 to 25 bar, such as in the range of 8 to 20 bar, such as in the range of 10 to 15 bar.

In an embodiment of the invention, biochar is added to the liquid biomass digestate. Biochar is well-known and may be produced in a number of different ways, for example by means of pyrolysis. In the context of the present invention, the pyrolysis may even use the solid digestate fraction or a fraction thereof as input. The addition of biochar may advantageously increase the efficiency of the microbial electrolysis cell processing.

According to an advantageous embodiment of the invention, the microbial electrolysis cell reactor is a two-chamber microbial electrolysis cell comprising a membrane.

By including a membrane, the microbial electrolysis cell produces hydrogen gas. It is noted that some methane may also be produced as a biproduct when the microbial electrolysis cell produces hydrogen gas. Carbon dioxide is also produced as from the anodic compartment. Other minor gas components may be produced.

According to an advantageous embodiment of the invention, the microbial electrolysis cell reactor is a one-chamber microbial electrolysis cell.

By excluding a membrane, the microbial electrolysis cell produces methane. Additionally, carbon dioxide will be produced when producing methane, although substantial amounts of this may be converted to methane. Other minor gas components may be produced.

According to an advantageous embodiment of the invention, said microbial electrolysis cell processing is performed in at least one microbial electrolysis cell reactor.

In some embodiments two or more microbial electrolysis cell reactors may be used in parallel, e.g. to obtain the desired scale at a given processing site.

In some embodiments two or more microbial electrolysis cell reactors may be used in series, e.g. two subsequent microbial electrolysis cell reactors, which may provide for an improved production of methane and/or hydrogen gas.

According to an advantageous embodiment of the invention, the microbial electrolysis cell processing comprising applying a voltage of no more than 1.8 volt, such as no more than 1.5 volt, such as no more than 1.23 volt.

In an embodiment of the invention, the microbial electrolysis cell (MEC) processing comprising applying a voltage in the range of 0.114 to 1.8 volt, such as in the range of 0.5 to 1.5 volt, such as in the range of 0.8 to 1.23 volt.

In an embodiment of the invention, the microbial electrolysis cell (MEC) processing comprising applying a voltage in the range of 0.114 to 1.8 volt, such as in the range of 0.114 to 1.5 volt, such as in the range of 0.114 to 1.23 volt.

According to an advantageous embodiment of the invention, the microbial electrolysis processing is performed in at least one microbial electrolysis cell reactor having a capacity of at least 5 m3, such as at least 20 m3, such as at least 100 m3.

In an embodiment of the invention, the microbial electrolysis processing is performed in at least one microbial electrolysis cell reactor having a capacity of 5-1,000 m3, such as 20-500 m3, such as 100-200 m3.

In some embodiments, two or more microbial electrolysis cell reactors may be used, e.g. in parallel, each of which may have the above defined capacity.

In one embodiment, the two or more microbial electrolysis cell reactors having a total capacity of 500-4,000 m3 are used.

According to an advantageous embodiment of the invention, the microbial electrolysis processing is performed in at least one continuous microbial electrolysis cell reactor having a capacity of at least 1 m3 per hour, such at least 5 m3 per hour, such as at least 20 m3 per hour.

In an embodiment of the invention, the microbial electrolysis processing is performed in at least one continuous microbial electrolysis cell reactor having a capacity of 1-50 m3 per hour, such 5-40 m3 per hour, such as 20-40 m3 per hour.

In some embodiments, two or more microbial electrolysis cell reactors may be used, e.g. in parallel, each of which may have the above defined capacity.

According to an advantageous embodiment of the invention, the method comprises adding a high energy biomass fraction to the biomass digestate before and/or during said microbial electrolysis cell processing.

An advantage of the above embodiment may be that the efficiency of the microbial electrolysis cell processing may be improved, e.g. by adding a high energy biomass fraction having a COD of at least 50,000 mg O₂/L, such as at least 100,000 mg O₂/L. In example embodiments, the high energy biomass fraction has a COD of 50,000-1,000,000 mg O₂/L, such as 100,000-1,000,000 mg O₂/L.

According to an advantageous embodiment of the invention, the separation comprises a screw press separation step.

According to an advantageous embodiment of the invention, the separation comprises a decanter centrifuge separation step.

In an embodiment of the invention, the decanter centrifuge separation step is performed on the liquid output of a screw press separation step.

According to an advantageous embodiment of the invention, the separation comprises a filter separation step.

In an embodiment of the invention, the filter separation step is performed on the liquid output of a decanter centrifuge separation step.

In an embodiment of the invention, the separation comprises one or more of a belt press separation step, a sedimentation step, a filter chamber press separation step, a screw press separation step, a decanter centrifuge step, and a filter separation step.

According to an advantageous embodiment of the invention, the liquid digestate fraction has a dry matter content of no more than 10% by weight of the liquid digestate fraction, such as less than 7% by weight of the liquid digestate fraction, such as less than 5% by weight of the liquid digestate fraction.

According to an advantageous embodiment of the invention, the liquid digestate fraction has a volatile solid content of at least 0.5% by weight of the liquid digestate fraction, such as at least 1% by weight of the liquid digestate fraction, such as at least 2% by weight of the liquid digestate fraction.

According to an advantageous embodiment of the invention, the solid digestate fraction has a water content of no more than 85% by weight of the solid digestate fraction, such as no more than 80% by weight of the solid digestate fraction.

According to an embodiment of the invention, the solid digestate fraction has a water content of 10-85% by weight of the solid digestate fraction, such as 30-85% by weight of the solid digestate fraction, such as 50-80% by weight of the solid digestate fraction.

According to an advantageous embodiment of the invention, the biomass has a dry matter content of at least 5% by weight of the biomass, such as at least 10% by weight of the biomass.

In an embodiment of the invention, the biomass has a water content below 95%.

In an embodiment of the invention, the biomass has a COD content of at least 20,000 mg/L, such as at least 40,000 mg/L, such as at least 60,000 mg/L.

In an embodiment of the invention, the biomass has a COD content of 20,000-200,000 mg/L, such as 40,000-150,000 mg/L, such as 60,000-100,000 mg/L.

According to an advantageous embodiment of the invention, the biomass comprises farm-based components, such as animal feces containing fractions and/or crop fractions.

Animal feces fractions may for example include manure and/or deep litter. Crop fractions may e.g. include aftercrop components.

According to an advantageous embodiment of the invention, the biomass comprises fibrous biomass, such as fibrous biomass having a particle size of at least 1 cm, such as at least 2 cm.

In an embodiment of the invention, the biomass comprises fibrous biomass, such as fibrous biomass having a particle size of 1-20 cm, such as 2-15 cm.

In the context of fibrous biomass, the term “particle size” is understood as a longest dimension of the particles in the biomass. Therefore, fibrous biomass having a particle size of at least 1 cm refers to fibrous biomass of particles having a length of at least 1 cm in at least one dimension. This may be measured e.g. by using a sieving tower.

According to an advantageous embodiment of the invention, the biomass comprises fibrous biomass having a particle size of at least 1 cm in an amount of at least 2% by weight of the biomass, such as at least 5% by weight of the biomass, such as at least 10% by weight of the biomass.

In an embodiment of the invention, the biomass comprises fibrous biomass having a particle size of at least 1 cm in an amount of 2-40% by weight of the biomass, such as 5-30% by weight of the biomass, such as 10-20% by weight of the biomass.

In an embodiment of the invention, the biomass comprises fibrous biomass having a particle size of 1-15 cm in an amount of 2-40% by weight of the biomass, such as 5-30% by weight of the biomass, such as 10-20% by weight of the biomass.

Typical fibrous biomasses and fibrous biomasses containing fractions in the above context may include straw, deep litter, hay, corn straw, grass, and any combination thereof.

In an advantageous embodiment of the invention, the biomass comprises one or more selected from the group consisting of straw, deep litter, hay, corn straw, grass, and any combination thereof.

In an advantageous embodiment of the invention, the biomass comprises straw.

In an embodiment of the invention, the biomass is not wastewater sludge.

In an embodiment of the invention, the biomass comprises wastewater sludge in an amount of no more than 10% by weight of the biomass, such as no more than 5% by weight of the biomass, or is free of wastewater sludge.

In an advantageous embodiment of the invention, the biomass comprises a content of animal feces and bedding material of at least 50% by weight of the biomass, such as at least 60% by weight of the biomass, such as at least 70% by weight of the biomass.

In an embodiment of the invention, the biomass comprises a total content of animal feces and bedding material of 50 to 100% by weight of the biomass, such as 60 to 90% by weight of the biomass, such as 70 to 80% by weight of the biomass.

According to an advantageous embodiment of the invention, the biomass is received in an input storage container.

In embodiments where the biomass is continuously fed to the at least one anaerobic digestion reactor, the anaerobic digestion reactor and the storage container may preferably be connected by a suitable piping system, including one or more pumps for continuously feeding the biomass.

According to an advantageous embodiment of the invention, the biomass is loaded into the storage container in a batchwise manner.

According to an advantageous embodiment of the invention, at least 1% by weight of the biomass in the at least one anaerobic digestion reactor is replaced per day, such as at least 2% by weight of the biomass, such as at least 3% by weight of the biomass.

In an embodiment of the invention, between 1 and 20% by weight of the biomass in the at least one anaerobic digestion reactor is replaced per day, such as between 2 and 15% by weight of the biomass, such as between 3 and 10% by weight of the biomass.

According to an advantageous embodiment of the invention, the method further comprises storing an output digestate of the microbial electrolysis cell processing in an output digestate storage.

According to an advantageous embodiment of the invention, the anaerobic digestion reactor has a capacity of at least 5 m3, such as at least 50 m3, such as at least 200 m3.

In an embodiment of the invention, the anaerobic digestion reactor has a capacity of 5-16,000 m3, such as 50-10,000 m3, such as 200-5,000 m3.

When both a first anaerobic digestion reactor and a second anaerobic digestion reactor are employed, each may have a capacity as stated above.

According to an advantageous embodiment of the invention, the method further comprises initializing the anaerobic digestion reactor by injecting a liquid digestate.

The initializing step may also be referred to as inoculation.

According to an advantageous embodiment of the invention, the method further comprises upgrading the biogas by reducing the content of carbon dioxide in the biogas.

In an embodiment of the invention, the upgrading comprises one or more purification steps based on water scrubbing, pressure swing adsorption, solvent adsorption, membrane filtration, amine gas treating, and methanation hereunder biomethanation.

Solvent adsorption may e.g. be based on dimethyl ether of polyethylene glycol, such known as the Selexol process.

Especially in embodiments where the product gas consists of biogas, the biogas obtained from the anaerobic digestion may be subjected to methanation, such as biomethanation. This may comprise using hydrogen gas produced in the microbial electrolysis cell for converting carbon dioxide in the biogas to methane.

According to an advantageous embodiment of the invention, the upgrading further comprises reducing the content of at least one of hydrogen sulfide, water, and carbon monoxide.

In an embodiment of the invention, the method further comprises pumping the biomass into the anaerobic digestion reactor by a biomass pump.

In an embodiment of the invention, the biomass pump has a pump flow of at least 0.2 m3 per hour, such as at least 1 m3 per hour. As an example, the biomass pump has a pump flow of 0.2-34 m3 per hour, such as 1-20 m3 per hour.

In an embodiment of the invention, the method further comprises pumping the biomass digestate into the solid liquid separator by a biomass digestate pump.

In an embodiment of the invention, the biomass digestate pump has a pump flow of at least 0.2 m3 per hour, such as at least 1 m3 per hour. As an example, the biomass digestate pump has a pump flow of 0.2-34 m3 per hour, such as 1-20 m3 per hour.

In an embodiment of the invention, the method further comprises pumping the liquid digestate fraction into the microbial electrolysis cell reactor by a liquid digestate pump.

In an embodiment of the invention, the liquid digestate pump has a pump flow of at least 0.2 m3 per hour, such as at least 1 m3 per hour. As an example, the liquid digestate pump has a pump flow of 0.2-34 m3 per hour, such as 1-20 m3 per hour.

In an embodiment of the invention, the method further comprises pumping the output digestate into an output digestate storage by an output digestate pump.

In an embodiment of the invention, the output digestate pump has a pump flow of at least 0.2 m3 per hour, such as at least 1 m3 per hour. As an example, the output digestate pump has a pump flow of 0.2-34 m3 per hour, such as 1-20 m3 per hour.

In an embodiment of the invention, the method further comprises a step of error state monitoring.

In an embodiment of the invention, the error state monitoring comprises measuring one or more parameters selected from dry matter content change, pH, temperature, biogas composition, and volatile fatty acid content in the digestate. Here, biogas composition and volatile fatty acid content in the digestate may be measured by means of gas chromatography and/or high-performance liquid chromatography.

In an embodiment of the invention, the error state monitoring is connected to measure on the anaerobic digestion reactor and/or the microbial electrolysis cell reactor.

According to an advantageous embodiment of the invention, the biogas produced from the anaerobic digestion has a concentration of methane gas of 30-65% by volume of the biogas, such as 40-65% by volume of the biogas, such 50-65% by volume of the biogas.

According to an advantageous embodiment of the invention, the microbial electrolysis cell processing produces a first product gas stream comprising methane gas, and optionally a second product gas stream comprising hydrogen gas, wherein the first product gas stream comprises a concentration of methane gas of at least 65% by volume of the product gas, such as at least 68% by volume of the product gas.

In an embodiment of the invention, the microbial electrolysis cell processing produces a first product gas stream comprising methane gas, and optionally a second product gas stream comprising hydrogen gas, wherein the first product gas stream has a concentration of methane gas in an amount of 65-80% by volume of the first product gas, such as 68-75% by volume of the first product gas.

According to an advantageous embodiment of the invention, the biogas produced from the anaerobic digestion comprises methane gas in a first concentration, wherein the microbial electrolysis cell processing produces a first product gas stream comprising methane gas, and optionally a second product gas stream comprising hydrogen gas, wherein the first product gas stream comprises methane gas in a second concentration, and wherein the second concentration exceeds the first concentration by at least 5 percentage points, such as at least 7 percentage points, such as at least 9 percentage points.

The above embodiment may be especially relevant for embodiments employing single chamber microbial electrolysis cell processing.

According to an embodiment of the invention, the biogas produced from the anaerobic digestion comprises methane gas in a first concentration, wherein the microbial electrolysis cell processing produces a first product gas stream comprising methane gas, and optionally a second product gas stream comprising hydrogen gas, wherein the first product gas stream comprises methane gas in a second concentration, and wherein the second concentration exceeds the first concentration by 5 to 40 percentage points, such as 5 to 25 percentage points, such as 7 to 20 percentage points, such as 9 to 15 percentage points.

The invention further relates to a system for producing product gas comprising methane, the system comprising
an anaerobic digestion reactor arranged producing biogas and a biomass digestate,
a solid liquid separation unit arranged to separate the biomass digestate into a liquid digestate fraction and a solid digestate fraction, and
a microbial electrolysis cell reactor arranged to produce methane and/or hydrogen gas.

In an advantageous embodiment of the invention, the system is configured to operate in accordance with the method of the invention or any of its embodiments.

### FIGURES

The invention will now be described with reference to the figures, where

FIG. 1A illustrates a method of producing product gas PG comprising methane according to an embodiment of the invention,

FIGS. 2A and 2B illustrates microbial electrolysis cell reactors MECR according to embodiments of the invention,

FIG. 3 illustrates an anaerobic digestion reactor ADR according to an embodiment of the invention,

FIGS. 4A-4D illustrate cavitation treatment according to embodiments of the invention,

FIG. 6 illustrates a measured current in two systems with the applied potential for example 4,

FIG. 7 illustrates methane and hydrogen production of example 6,

FIG. 8 illustrates methane production of example 7,

FIG. 9 illustrates methane production of example 8,

FIG. 10 illustrates methane production with ultrasonicated liquid digestate of example 9, and

FIG. 11 illustrates methane production with post-treated biomass liquid fraction of example 10.

### DETAILED DESCRIPTION

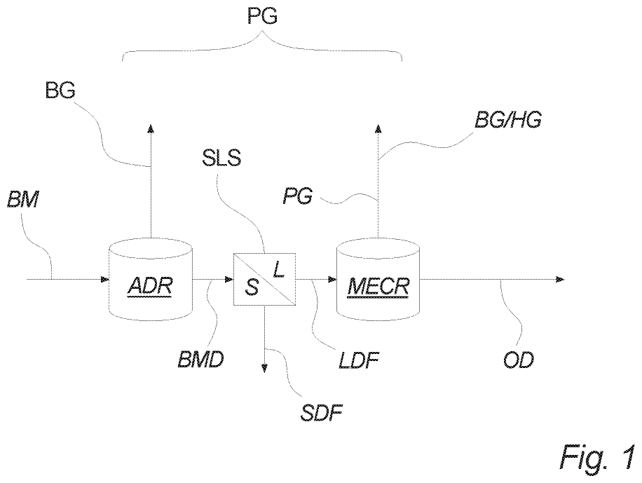

Referring to FIG. 1, a method of producing product gas PG comprising methane according to an embodiment of the invention and a corresponding system for producing product gas PG comprising biogas according to an embodiment of the invention are described.

First, the provided biomass BM is fed into the anaerobic digestion reactor ADR. In some embodiments, the biomass BM is continuously fed into the anaerobic digestion reactor ADR. In other embodiments, the biomass BM is fed into anaerobic digestion reactor ADR at distinct time periods, such as distinct time periods each day. Still in further embodiments, the biomass BM is fed into anaerobic digestion reactor ADR to reach a desired level of biomass BM, whereafter the biomass BM is subjected to anaerobic digestion in the anaerobic digestion reactor ADR. In embodiments where the resulting biomass digestate BMD is continuously fed out of the anaerobic digestion reactor ADR, the rate of which biomass digestate is removed from the anaerobic digestion reactor ADR may typically be set based on the rate with which the biomass BM is fed into the anaerobic digestion reactor ADR.

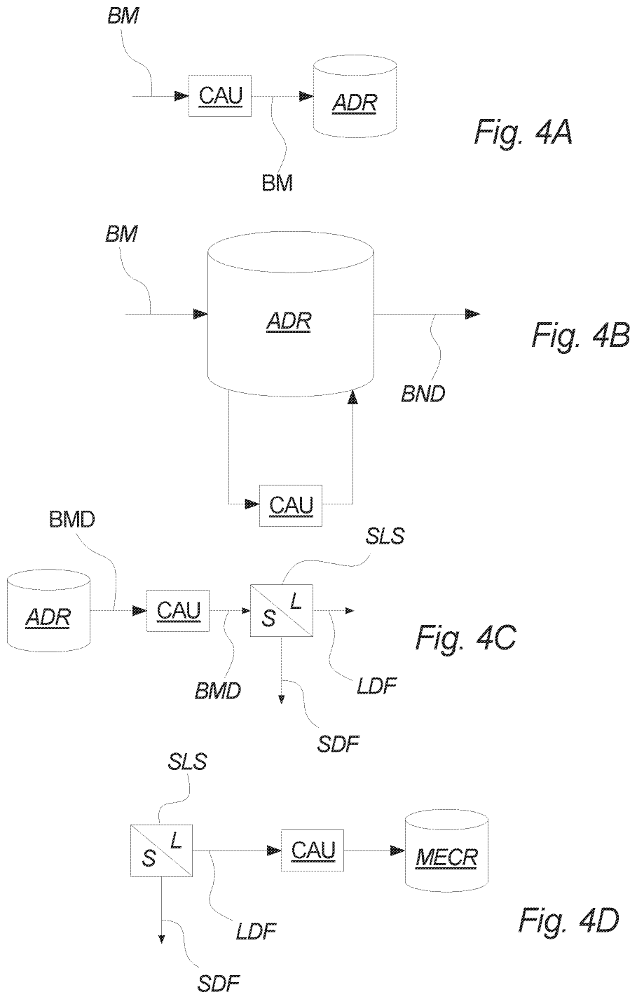

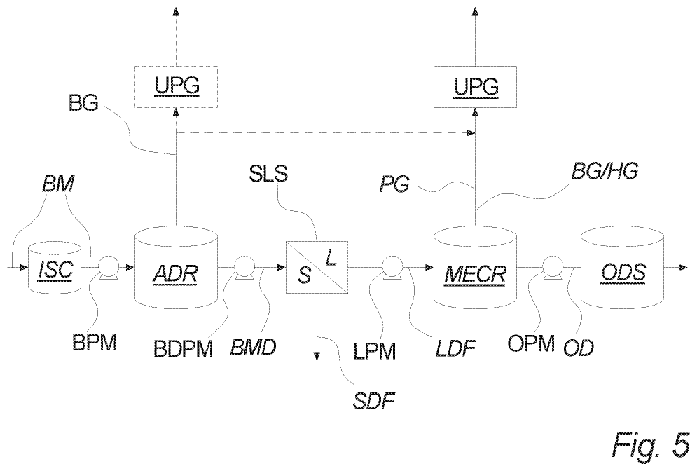

Thus, the biomass is subjected to anaerobic digestion for a period of time, which is straight forward for batch-based processes. For continuous processes, the retention time typically refers to the average retention time, which is determined by the feeding rates and the capacity of the anaerobic digestion reactor ADR. Typically, the retention time is a pre-determined period of time, but it may also be at least partly based on measured values related to one or more of the anaerobic digestion reactor ADR, the biomass BM, the biomass digestate BMD, and the produced biogas. An anaerobic digestion reactor ADR as described in relation to FIG. 4 is suitable for use in the method illustrated on FIG. 1.

During the anaerobic digestion of the biomass BM, methane is produced as biogas BG in the anaerobic digestion reactor ADR. The biogas BG may be collected e.g. by a suitable piping system for further processing and/or transport. In many embodiments, the collection may be performed continuously, i.e. such that no substantial buildup of product gas occurs. Typically, there may be a small capacity to temporarily build up product gas, e.g. by using a flexible membrane. This may e.g. serve to even out the rate with which product gas is collected from the anaerobic digestion reactor ADR.

The anaerobic digestion reactor is sealed from ambient surroundings, whereby anaerobic conditions may be ensured by minimizing oxygen presence. Additionally, the sealing may help to contain the produced the biogas until collection.

The output of the anaerobic digestion is, besides the biogas, a biomass digestate, which is fed to a solid liquid separation unit SLS for separation into a solid digestate fraction SDF and a liquid digestate fraction LDF.

Optionally, the biomass digestate BMD may be subjected to cavitation treatment, as described in relation to FIGS. 4A-4D. The embodiments illustrated in FIGS. 4A-4D may also be combined to include two or more cavitation treatment steps.

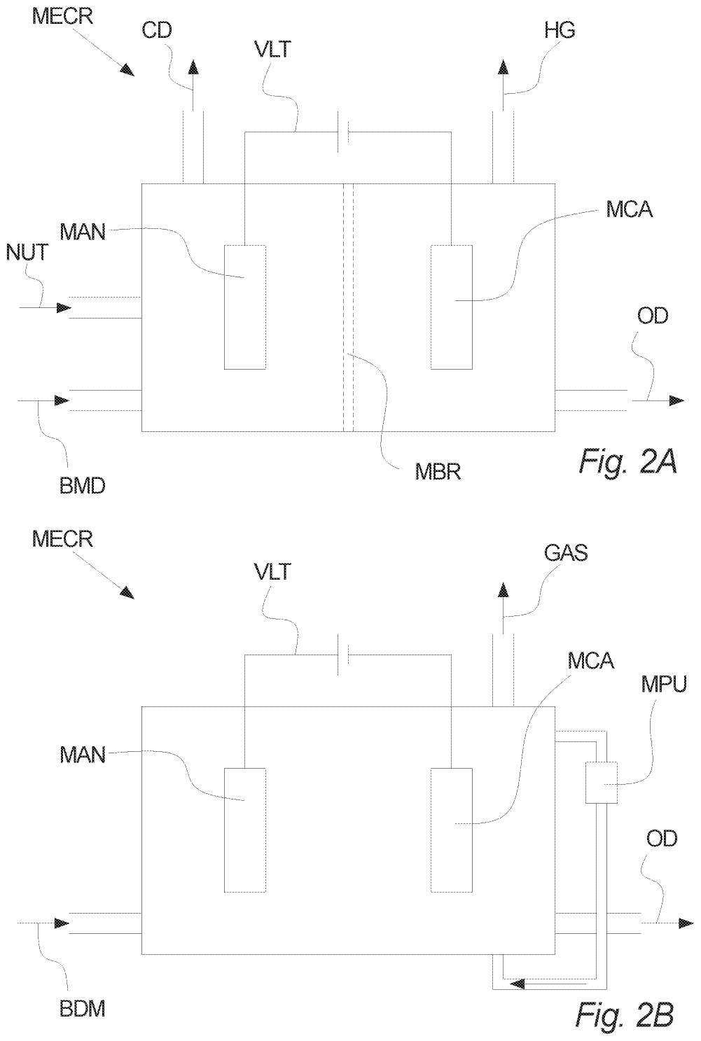

The liquid digestate fraction is then fed to a microbial electrolysis cell reactor MECR for microbial electrolysis cell processing. Depending on the configuration of the microbial electrolysis cell reactor MECR, particularly whether this is configured as a single chamber microbial electrolysis cell reactor MECR or a dual chamber microbial electrolysis cell reactor MECR with a separating membrane, the microbial electrolysis cell processing will produce biogas BG and/or hydrogen gas HG. Additionally, the output of the MECR, further to the biogas BG and/or hydrogen gas, is an output digestate OD. The microbial electrolysis cell reactors MECR described in the context of FIGS. 2A and 2B may be used.

As illustrated the biogas BG produced in the anaerobic digestion reactor ADR and the biogas BG and/or hydrogen gas produced by the microbial electrolysis cell reactor MECR is referred to as product gas PG. Thus, depending on the configuration of the microbial electrolysis cell reactor MECR, the product gas PG may consist essentially of biogas BG, or contain biogas BG and hydrogen gas HG. The processing of the product gas PG including biogas BG and optionally hydrogen gas HG is exemplified in the embodiments described in relation to FIG. 5.

In some embodiments, only a fraction of the liquid digestate fraction is fed to the microbial electrolysis reactor MECR.

Referring to FIGS. 2A and 2B, microbial electrolysis cell reactors MECR according to embodiments of the invention are described.

The microbial electrolysis cell reactor MECR comprises an anode MAN and a cathode MCA, which in the embodiment of FIG. 2A is separated by a membrane MBR.

The anode MAN may comprise or be made from a number of different materials, including but not limited to carbon (e.g. in the form of carbon cloth, carbon paper, carbon felt, carbon foam, biochar, glassy carbon, carbon nanotube sponges, etc.), graphite (e.g. in the form of graphite felt, graphite granules, graphite brushes), conductive polymer-based composite material (e.g. using polymers such as polyaniline, polypyrrole, polythiophene, poly-co-o-aminophenol, etc.), metals and metal oxides, graphene derivatives with metal/metal oxide nanoparticles or conductive polymer-based composite materials.

The cathode MCA may comprise or be made from a number of different materials, including but not limited to carbon-based materials, composites, metals and metal oxides. Generally, similar material as for the anode may be used. Conductive materials are used to make electrodes, such as platinum meshes, carbon felt, carbon fibre, and carbon cloth. Catalysts, such as platinum and titanium, may be used to enhance performance of the cathode.

Biomass digestate BMD is injected to the microbial electrolysis cell reactor MECR through a suitable inlet and collected as output digestate OD by a suitable outlet after processing. It is noted that the specific configuration with respect to inlets and outlets for the biomass digestate BMD and output digestate OD may differ between specific embodiments.

As shown in FIG. 2A, the microbial electrolysis cell reactor MECR may have an inlet for adding nutrients NUT. Such nutrients may typically be added e.g. to balance macronutrients and/or micronutrients or may add fractions comprising high energy content accessible for the applied microbial cultures, e.g. signified by a high COD.

In some embodiments, the nutrient NUT inlet is disposed with, as illustrated in with FIG. 2B.

The microbial electrolysis cell reactor MECR may further comprise one or more outlets for collecting gas produced during the microbial electrolysis cell processing. In two-chamber embodiments, two outlets may typically be used, one for collecting carbon dioxide CD from the anode and another outlet for collecting hydrogen gas HG from the cathode.

The membrane MBR as illustrated in FIG. 2A is configured to allow passage of protons produced by microorganism present at the anode. The passing protons are reduced at the cathode MCA due to a sufficiently high voltage VLT being applied. Thereby, hydrogen gas is formed, which may then be collected.

Additionally, carbon dioxide is formed at the anode by the microorganisms in the same reaction as the protons. The carbon dioxide may then be collected besides the hydrogen gas as the two main constituents gasses produced by the microbial electrolysis cell MECR.

In the embodiment illustrated in FIG. 2B, no membrane is applied, and therefore carbon dioxide may therefore exist at the cathode, whereby methanogenic microorganisms may produce methane and water from the hydrogen and carbon dioxide.

It is noted that depending on the specific design, membrane used, etc. the produced gas may contain both hydrogen and methane, however, the embodiments in FIGS. 2A and 23 serves to illustrate how to influence the production in the direction of hydrogen and methane, respectively.

Another aspect illustrated on FIG. 2B is that the microbial electrolysis cell reactor MECR further comprises a MEC pump MPU, which is arranged to pump biomass digestate BDM from one part of the microbial electrolysis cell reactor MECR to another part of the microbial electrolysis cell reactor MECR in a loop, whereby agitation in the biomass digestate BDM is introduced and any tendency for clogging may further be reduced. The pumping flow of the MEC pump MCU may be adjusted depending e.g. on the composition of the biomass digestate BDM. As an example, if the biomass digestate BDM having a higher tendency to introduce clogging is used, the pump flow may be increased to provide more agitation. It is noted that the use of a MEC pump MPU may be done independent on other aspects illustrated on FIG. 2B, and may also be usable with two-chamber MICs, such as the MEC illustrated in FIG. 2A.

It is noted that the design illustrated in FIGS. 2A and 2B is only a schematic representation, and that this may be implemented in a number of different ways. As an illustrative example, electrodes may e.g. comprise several plates in in certain forms, such as cylinder form. It is noted that depending on the specific design, membrane used, etc. the produced gas may contain both hydrogen and methane, however, the embodiments in FIGS. 2A and 2B serves to illustrate how to influence the production in the direction of hydrogen and methane, respectively.

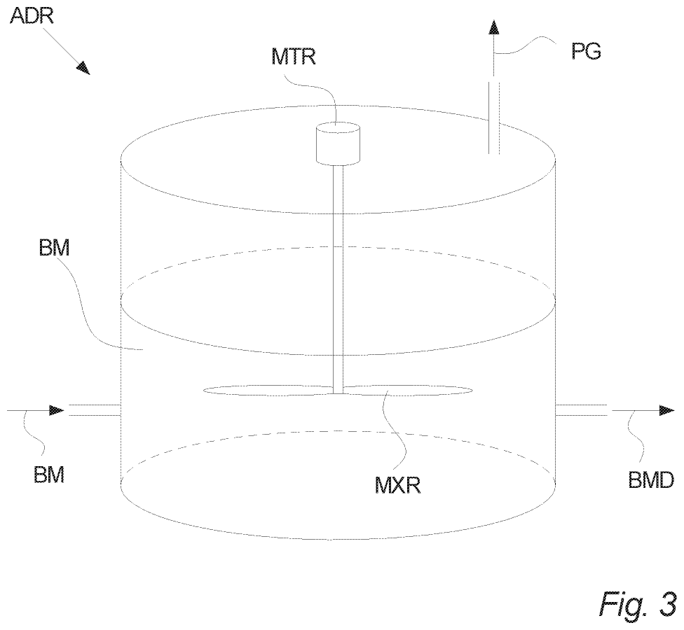

Referring to FIG. 3, an anaerobic digestion reactor ADR according to an embodiment of the invention is described.

As shown in FIG. 3, the biomass 3M is received through a suitable biomass inlet. After anaerobic digestion, the resulting biomass digestate BMD is ejected by a suitable outlet for further processing in the microbial electrolysis cell reactor MECR.

During the anaerobic digestion, the biomass BM may be agitated by a suitable mixer MXR brought into rotation by a motor MTR. Depending on the circumstances, such as the specific composition of the biomass BM, hereunder dry matter content, content of large particle sized fibrous biomass, etc., the rotational speed of the mixer MXR may be varied by the motor MTR.

Now, referring to FIGS. 4A-4D, the use of cavitation treatment according to different embodiments of the invention is described.

Generally, cavitation treatment may help to increase the processability of the biomass BM for anaerobic digestion or the biomass digestate BMD for the microbial electrolysis cell processing by physically breaking down the biomass into smaller particles and increasing the relative surface area.

Cavitation may e.g. be induced by mechanical treatment, e.g. by a fast rotating propeller or by suitable ultrasonic treatment.

First, on FIG. 4A, a cavitation unit CAU is shown initially, i.e. before the anaerobic digestion reactor ADR. The biomass BM may thus be treated by cavitation treatment prior to anaerobic digestion.

In some embodiments, depending on the capacity of the cavitation unit CAU relative to the flow rate of the biomass BM, only a part of the biomass may be treated in the cavitation unit CAU, and another part be fed around the cavitation unit CAU and directly into the anaerobic digestion reactor ADR. Alternatively, or in addition thereto, two or more cavitation units CAU may be installed for parallel treatment to increase the capacity of the cavitation treatment.

In FIG. 4B, the cavitation unit CAU is connected in a loop to the anaerobic digestion reactor ADR. A part of the biomass CM of anaerobic digestion reactor ADR may then continuously be fed into the loop and through the cavitation unit CAU and then back into the anaerobic digestion reactor ADR.

FIG. 4C illustrates another possibility of cavitation treatment, where a cavitation unit CAU is disposed after the anaerobic digestion reactor ADR, but before the solid liquid separator SLS. Due to the breakdown of fibrous biomass below the solid liquid separator SLS, the need for separation may in some cases be somewhat reduced, and may in some cases result in the number of solid liquid separation steps necessary being reduced. This may also apply for the embodiments illustrated in FIGS. 4A-4B, where the cavitation treatment is also employed before the solid liquid separator SLS.

Finally, in FIG. 4D, a cavitation unit CAU is disposed after separation and only on the liquid digestate fraction LDF. Even after the separation, the liquid digestate fraction LDF may still contain a content of biomass which is not easily processable in the microbial electrolysis cell reactor MECR. This processability of this content may then be significantly increased by the cavitation treatment.

Referring to FIG. 5, a method of producing product gas PG comprising methane according to an embodiment of the invention is described.

Further to the embodiment of FIG. 1, the FIG. 5 illustrates a number of aspects, which may be implemented in the embodiment of FIG. 1, either individually or collectively.

First, FIG. 5 illustrates the presence of a biomass pump BPM arranged to pump the biomass BM into the anaerobic digestion reactor ADR. Also, FIG. 5 illustrates the presence of a biomass digestate pump BMPM arranged to pump the biomass digestate BMD into the solid liquid separator SLS. Furthermore, FIG. 5 illustrates a liquid digestate pump LPM arranged to pump the liquid digestate fraction LDF into the microbial electrolysis cell reactor MECR. Also, FIG. 5 illustrates an output digestate pump OPM arranged to pump the output digestate into an output digestate storage ODS.

In some embodiments the output digestate pump OPM may pump the output digestate OD directly to be transported to different facilities e.g. to tank trucks for transport to a storage at a farm. In such cases, the output digestate storage ODS may in some cases be dispensed with. Also, when including an output digestate storage ODS, a further pump for pumping the output digestate OD from the output digestate storage ODS may be included.

In some embodiments, the biomass BM received may be stored in an input storage container ISC. Then, the biomass pump BPM draws biomass BM from the input storage container ISC. A further pump for pumping the biomass into the input storage container ISC may in some embodiments be used.

As illustrated in FIG. 5, one or more upgrading units UPG may also be provided to upgrade the produced gas. Especially when production of hydrogen gas HG is desired, it may be beneficial to handle the biogas BG produced in the anaerobic digestion reactor ADR and the hydrogen gas HG from the microbial electrolysis cell reactor MECR separately.

However, when production of methane is desired, it may be desirable to either configure the microbial electrolysis cell reactor MECR to produce further biogas BG comprising methane, or to use hydrogen gas HG from the microbial electrolysis cell reactor MECR to upgrade carbon dioxide from the biogas BG of the anaerobic digestion reactor ADR to figure methane.

### EXAMPLES

### Example 1—Anaerobic Digester Conditions

A mixture of biomasses comprising manure, deep litter, food waste and industrial waste products was subjected to a two-step anaerobic digestion process with residence time at 20 and 10 days for the first and second anaerobic digestion step, respectively.

The resulting biomass was subjected to a two-step solid-liquid separation, using first a screw press separation step on the biomass digestate and thereafter a decanter centrifuge separation step on the liquid fraction of the screw press separation step.

The production of liquid decanter centrifuge fraction was estimated at 60-66 m3/hour.

### Example 2—Liquid Digestate Fraction

The liquid decanter centrifuge fraction resulting from example 1 was analyzed.

TABLE 1
Analytic content of decanter liquid, TS and VS are based
on 10 different samples from a period of a year.

| TS | VS | COD | N | P | K | NH4—N | NH4—N | |
|---|---|---|---|---|---|---|---|---|
| [%] | [%] | [g/m³] | [g/kg] | [g/kg] | [g/kg] | [g/kg] | [%] | pH |
| 4.48 ± 0.26 | 2.87 ± 0.23 | 50,000 | 4.18 | 0.85 | 5.66 | 2.69 | 64 | 8.04 |

The above content shows a remaining content of solids (TS), including volatile solids (VS), which demonstrates the suitability of the liquid decanter centrifuge fraction for use in microbial electrolysis cell processing.

### Example 3—Maximum Theoretical Microbial Electrolysis Cell Processing

The liquid decanter centrifuge fraction resulting from example 1 was used as a liquid digestate fraction. To assess the output of a microbial electrolysis cell processing, a simulated microbial electrolysis cell processing was performed.

The simulation model was run with the following conditions/assumptions:
Two-chamber MEC
All VS content is degradable as glucose or acetate
Efficiency of MEC is set as conversion factor of decanter liquid
60 m3/hour liquid decanter centrifuge fraction is used

The degradation of glucose in a dual chamber MEC unit will correspond to a hydrolysis of glucose, which will form CO₂ and H₂.

C₆H₁₂O₆+2H₂O→6CO₂+12H₂

This equation shows a formation of 6 moles of CO₂ and 12 moles of H₂ per mole of glucose. From this equation the outcome per glucose unit can be found.

$${Yield}_{{CO}_{2}} = \frac{6 {mol}_{{CO}_{2}}}{1 {mol}_{glucose}} * 44 \frac{{g}_{{CO}_{2}}}{{mol}_{{CO}_{2}}} * \frac{1 {mol}_{glucose}}{180 {g}_{glucose}} = 1.47 \frac{{g}_{{CO}_{2}}}{{g}_{glucose}}$$

$${Yield}_{{H}_{2}} = \frac{12 {mol}_{{H}_{2}}}{1 {mol}_{glucose}} * 2 \frac{{g}_{{H}_{2}}}{{mol}_{{H}_{2}}} * \frac{1 {mol}_{glucose}}{180 {g}_{glucose}} = 0.13 \frac{{g}_{{H}_{2}}}{{g}_{glucose}}$$

Since CO₂ and H₂ are produced as gasses, it is most relatable to have the yields in volume and not mass.

$${Yield}_{{CO}_{2}} = 1.47 \frac{{g}_{{CO}_{2}}}{{g}_{glukose}} * \frac{1 {L}_{{CO}_{2}}}{1.98 {g}_{{CO}_{2}}} = 0.74 \frac{{L}_{{CO}_{2}}}{{g}_{glucose}}$$

$${Yield}_{{H}_{2}} = 0.13 \frac{{g}_{{H}_{2}}}{{g}_{glukose}} * \frac{1 {L}_{{H}_{2}}}{0.089 {g}_{{H}_{2}}} = 1.498 \frac{{L}_{{H}_{2}}}{{g}_{glucose}}$$

From the yields, the molar ratio is observed from the equation. For every mole of CO₂ produced, two moles of H₂ are produced. The yields can be used to see the production from decanter liquid. The condition of 60 m³ decanter liquid per hour is used, an estimated of an efficiency of 72.5% is used according to studies on hydrogen formation from acetate. It is estimated that 1 gram of VS equals 1 gram of glucose. This will give the production of:

$${CO}_{2} , : 26 , 900 \frac{{g}_{VS}}{{m}_{decanter}^{3}} * 60 \frac{{m}_{decanter}^{3}}{h} * 72.5 {\%}_{Efficiency} * 0.74 \frac{{L}_{{CO}_{2}}}{{g}_{glucose}} * {10}^{- 3} \frac{{L}_{{CO}_{2}}}{{m}_{{CO}_{2}}^{3}} = 867 \frac{{m}_{{CO}_{2}}^{3}}{h}$$

$${H}_{2} : 26 , 900 \frac{{g}_{VS}}{{m}_{decanter}^{3}} * 60 \frac{{m}_{decanter}^{3}}{h} * 72.5 {\%}_{Efficiency} * 1.498 \frac{{L}_{{H}_{2}}}{{g}_{glucose}} * {10}^{- 3} \frac{{L}_{{H}_{2}}}{{m}_{{H}_{2}}^{3}} = 1 , 754 \frac{{m}_{{H}_{2}}^{3}}{h}$$

The production of gasses from a MEC on the decanter liquid will yield 1,754 m_{H}_{₂}³/h and 867 m_{CO}_{₂}³/h. This will correspond to a yearly production of 15.4*10⁶ m_{H}_{₂}³, and 7.6*10⁶ m_{CO}_{₂}³

The simulation was repeated for a one chamber MEC.

The equation for methanation is:

4H₂+CO₂→CH₄+2H₂O

This process is not without a loss and is estimated to have an efficiency of 95%, which would utilize all hydrogen but produce 417 m_{CH}_{₄}³ and 451 m_{CO}_{₂}³/h. This would be a yearly production of 3.65 Mm_{CH}_{₄}³ and 3.95 Mm_{CO}_{₂}³.

The above simulations show how considerable amounts of hydrogen gas or methane gas may be produced from the liquid digestate fraction resulting from anaerobic digestion.

### Example 4—Potential for Microbial Electrolysis Cell Processing in Liquid Decanter Centrifuge Fraction

Filtered liquid digestate was used to represent the liquid decanter centrifuge fraction resulting from example 1 as input to lab-scale microbial electrolysis cell processing.

Reactor configuration: A bulk electrolysis cell with a capacity of 75 mL of sample solution was used in the experiment. The working electrode was made of reticulated vitreous carbon (RVC) and the auxiliary electrode was a coiled 23 cm platinum wire within a fritted glass isolation chamber. A RE-5B Ag/AgCl reference electrode was used. A potentiostat was used as power source.

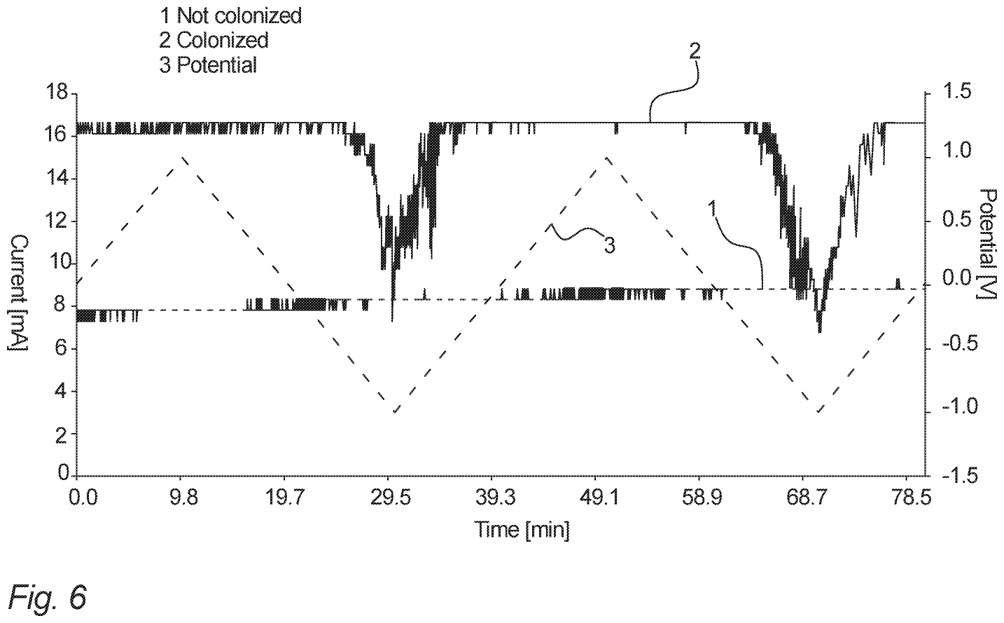

Experiment: The working electrode was inoculated with 300 ml of filtered liquid digestate for 7 days at 37 degrees Celsius in a non-sealed container to allow produced biogas to escape the reactor. After two days of inoculation, the reactor was fed with a cellulose solution. The colonised electrode was subsequently placed inside the MEC reactor, and the reactor was filled with a fresh batch of filtered liquid digestate. A non-colonized electrode was used in another MEC reactor with filtered liquid digestate, which acted as a reference system. Cyclic voltammetry was performed using 5 segments with an upper potential of 1 V, a lower potential of −1 V and a sweep rate of 0.1 V/s. The measured current in the two systems are shown in FIG. 6 together with the applied potential. In FIG. 6, figure reference 1 corresponds to the non-colonized electrode, figure reference 2 corresponds to the colonized electrode, and figure reference 3 corresponds to the potential.

It is clearly seen that the colonized electrode allows for a significantly increased current flow as compared to the non-colonized electrode, indicating the presence of electroactive microbial activity. It is further noted that the maximum voltage used (1 V) is below the minimum voltage required for water electrolysis, showing that MEC processing of the liquid decanter centrifuge fraction can produce methane and/or hydrogen gas using less external energy compared to conventional electrolysis.

### Example 6 Hydrogen and Methane Gas Production from Dual Chamber Microbial Electrolysis Cell Reactor

### Reactor Configuration:

A dual chamber microbial electrolysis reactor, with a total capacity of 200 mL in each chamber and separated by Nafion N117 cation exchange membrane, was used for the experiment. Both anode and cathode were made of carbon felt, each with a surface area of 38 cm

### Experiment:

The anodic chamber was inoculated with 150 g of liquid digestate fraction while 150 g of 0.1 M sodium chloride was added to the cathodic chamber. A cell potential of 0.8 V was applied to the reactor via a power supply and the current was recorded by measuring the voltage drop across an external resistance of 1.3 Ohm. The reactors were gently stirred at 200 rpm and incubated at 30 degrees C. for 31 days with separate gas samples taken from the sealed headspace from both the anode and the cathode chambers. The control reactors were treated to the same conditions but without electrodes and without the addition of a cell voltage of 0.8 V. All treatments were performed in duplicates.

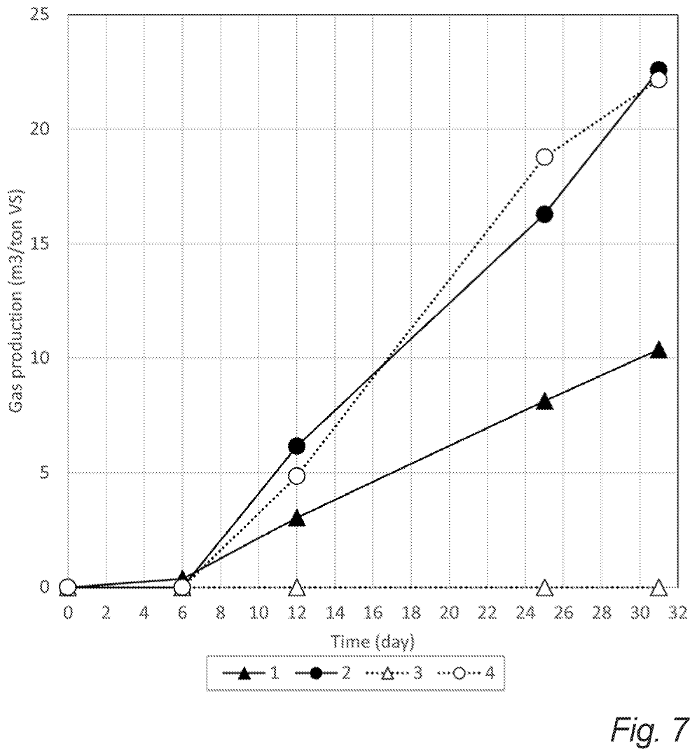

FIG. 7 shows results for methane and hydrogen production in dual chamber microbial electrolysis reactors containing liquid digestate fraction with and without the addition of 0.8V. Data from duplicate reactors. Legend: (1) Filled triangle, methane gas from control reactors, (2) filled circle, methane gas from reactors with addition of 0.8 V, (3) open triangle, hydrogen gas from control reactors, (4) open circle, hydrogen gas from reactors with addition of 0.8 V.

As seen in FIG. 7, the duplicate reactors supplied with 0.8 V (legend 4, open circle) produced up to 22.16 m³ H₂/ton VS from the cathodic chamber of the microbial electrolysis cell while none could be observed in the control reactors. Additionally, from the anodic chamber, the captured gas was approximately 2.17 times higher in methane (legend 2, filled circle) than the control reactors (legend 1, filled triangle). Overall, this represented an increase of 180% in energy gain (MJ/ton VS) in the microbial electrolysis reactors compared based on the enhanced production of both hydrogen and methane gas. It is also worth noting that the supplied voltage of 0.8 V is 1.5 times lower than the theoretical water hydrolysis voltage of 1.23 V. These results provide a strong proof-of-concept that a dual chamber microbial electrolysis cell with a membrane can be used to not only produce hydrogen gas but also at the same time enhance methane gas production from the liquid digestate fraction.

### Example 7 Methane Production from Single Chamber Microbial Electrolysis Cell Reactor

### Reactor Configuration:

The microbial electrolysis reactor consisted of a single chamber with a total capacity of 550 mL. Both anode and cathode, made of carbon felt with a surface area of 38 cm² were placed in the chamber.

### Experiment:

The microbial electrolysis cell was inoculated with 200 g of liquid digestate fraction. A cell potential of 0.8 V was applied to the reactor via a power supply and the current was recorded by measuring the voltage drop across an external resistance of 1.3 Ohm. The reactors were gently stirred at 200 rpm and incubated at 30 degrees C. for 25 days with gas samples taken from the sealed headspace. The control reactors contained the same volume of liquid digestate fraction but without electrodes and without the addition of a cell voltage of 0.8 V. All treatments were performed in duplicates.

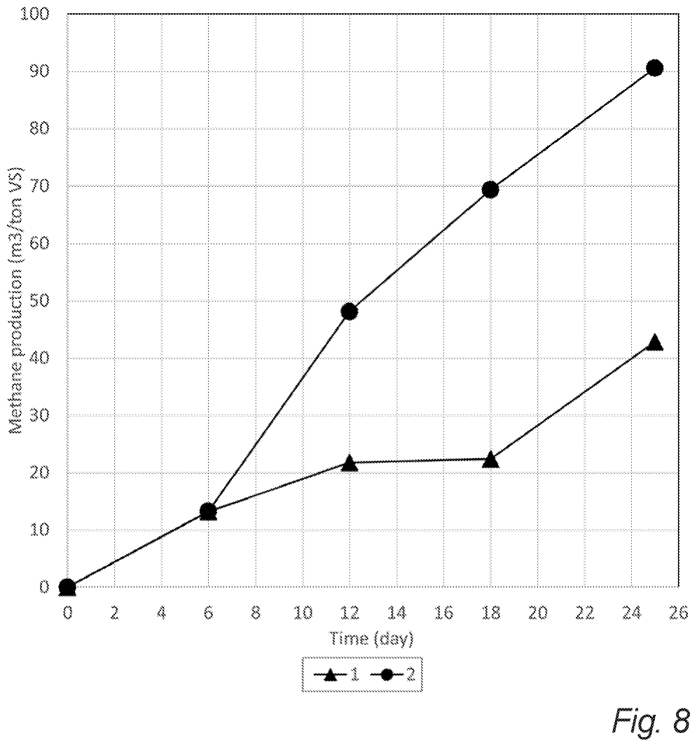

FIG. 8 shows results for methane production in single chamber microbial electrolysis reactors containing liquid digestate fraction with and without the addition of 0.8V. Data from duplicate reactors. Legend: (1) Filled triangle, methane gas from control reactors, (2) filled circle, methane gas from reactors with addition of 0.8 V.

In more detail, FIG. 8 illustrates the methane production in the microbial electrolysis reactors compared to the controls. An increased production of methane (legend 2: filled circle) is observed from day 6 onwards until it had produced 111% more methane than that of the control (legend 1: filled triangle) by the end of the experiment. Moreover, the biogas composition ratio of CH₄ to CO₂ in the anodic headspace of the microbial electrolysis cell reactors was approximately in the ratio of 69% CH₄ to 31% CO₂. This was higher compared to the ratios of 60% CH₄ to 40% CO₂ as seen in the biogas measured in the headspace of the control reactors. This not only represents an intrinsic biogas upgrading capability of the microbial electrolysis reactor but also attributes economical savings towards downstream CO₂ removal. These results contribute to the proof-of-concept that a single chamber microbial electrolysis cell can be used to produce enhanced methane gas from liquid digestate fraction.

### Example 8 Methane Production from Single Chamber Microbial Electrolysis Cell at Varying Applied Potential

The reactor configurations and experimental set up were similar to that described in example 7 and incubated at 30 degrees C. for a total of 31 days. The only notable difference was the supply of a higher cell voltage at 1.8 V.

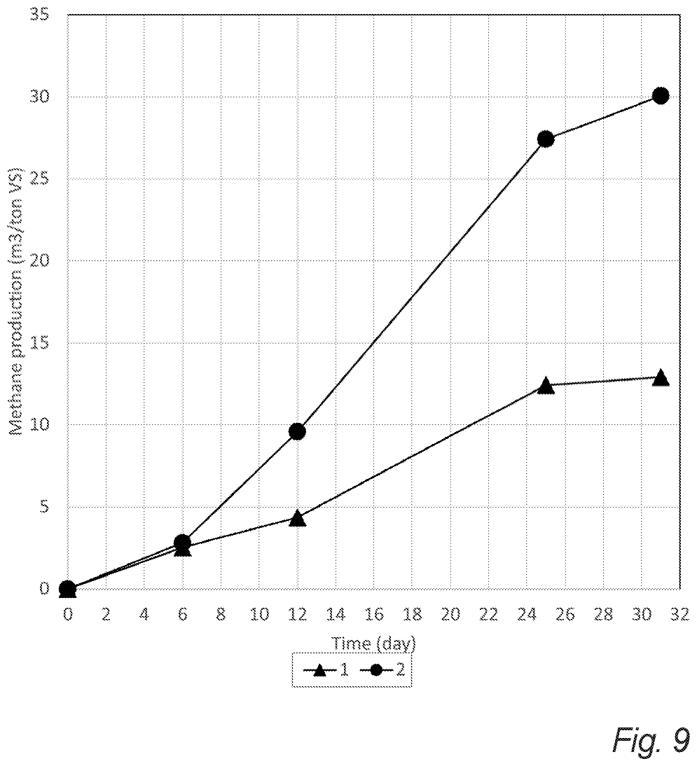

FIG. 9 shows results for methane production in single chamber microbial electrolysis reactors containing liquid digestate fraction with and without the addition of 1.8V. Data from duplicate reactors. Legend: (1) Filled triangle, methane gas from control reactors, (2) filled circle, methane gas from reactors with addition of 1.8 V.

In more detail. FIG. 9 depicts methane production in the single chamber microbial electrolysis cell reactors with and without the addition of 1.8 V. The reactors supplied with 1.8 V produced approximately 133% higher methane gas (legend 2: filled circle) by the end of the experimental period compared to the control (legend 1: filled triangle). These results showed that the microbial electrolysis cells can be used with varying voltage supply (0.8V or 1.8V) to enhance methane production from the liquid digestate fraction. It is worth noting that while the added voltage increased by 1 V between the two tested voltages, the methane production did not differ significantly among the two indicating a favorably reduced energy input to achieve the same outcome.

### Example 9 Methane Production in Single Chamber Microbial Electrolysis Cell Reactors with Liquid Digestate Fraction Pretreated by Ultrasonic Cavitation

The reactor configuration was similar to that described in example 7. For the experiment, the liquid digestate fraction was first pre-treated to ultrasonic cavitation in an ultrasonic bath (215 W, 35 kHz) for 30 minutes. Afterwards, 200 g of the ultra-sonicated liquid digestate fraction was transferred to single chamber microbial electrolysis reactors (n=4) as well as control reactors (n==2) and incubated for 31 days at 30 degrees C.

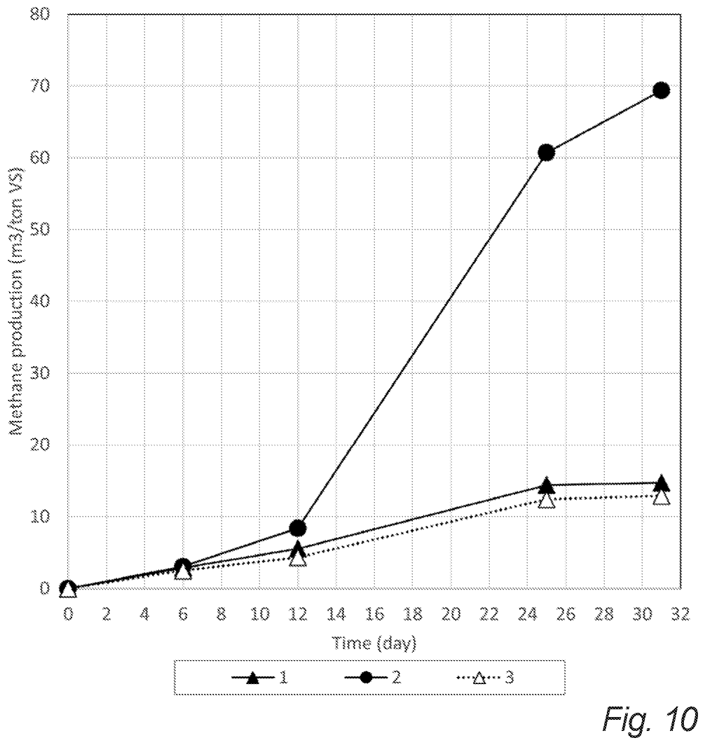

FIG. 10 shows methane production in single chamber microbial electrolysis reactors containing ultra-sonicated liquid digestate fraction with and without the addition of 0.8V. Data from duplicate control reactors and quadruplicate microbial electrolysis reactors. Legend: (1) Filled triangle, methane gas from control reactors of ultrasonicated liquid digestate fraction, (2) filled circle, methane gas from reactors with addition of 0.8 V, (3) open triangle, methane gas from control reactors of untreated liquid digestate fraction.

As per FIG. 10, the methane production was enhanced in the microbial electrolysis cell reactors with a supply of 0.8 V. The methane increase (legend 2: filled circle) was as high 372% more than that of the control (legend 1: filled triangle). It was assumed that the ultra-sonication treatment aided in the degradation of the organic matter via mechanical vibration, possibly resulting in a higher amount of easily degradable molecules which were then converted further to methane in the microbial electrolysis cells. Notably, the aforementioned ultrasonicating conditions alone were unremarkable as the methane increase (legend 1: filled triangle) compared to untreated controls (legend 3: open triangle) were marginal. These results highlight the synergistic effect of the microbial electrolysis cells with pretreatment options such as ultrasonic cavitation.

### Example 10 Methane Production in Single Chamber Microbial Electrolysis Cell Reactors with Liquid Digestate Fraction from Heat and Pressure Post-Treated Biomass Digestate

The reactor configuration was similar to that described in example 7. The biomass digestate was first subjected to heat and pressure post-treatment before the solid-liquid separation. The post-treatment was performed at 165 degrees C. for 30 minutes at an approximate pressure of 10 bars. A portion of biomass digestate was not subjected to these conditions to be used as control. Both portions were then centrifuged at 2600 rpm for 5 mins to produce the liquid digestate fraction to be transferred to the microbial electrolysis cells at 0.8 V.

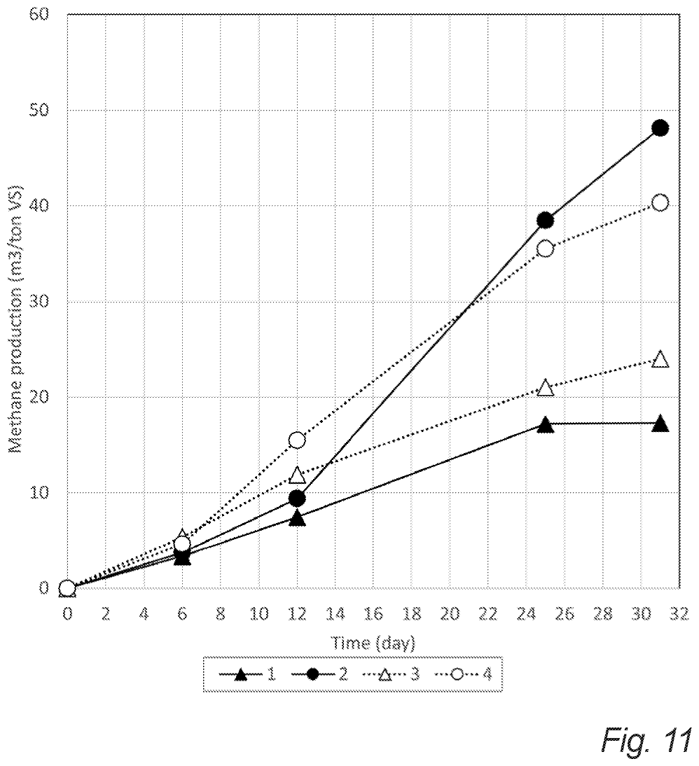

FIG. 11 shows methane production in single chamber microbial electrolysis reactors with and without the addition of 0.8V, containing liquid digestate fraction from heat and pressure treated biomass. Filled markers correspond to reactors containing post-treated biomass liquid fraction, open markers correspond to control biomass not subjected to post-treatment. Circles correspond to methane from reactors with 0.8 V, triangles correspond to control reactors with no added voltage.

As seen in FIG. 11, an increased methane production was observed in the microbial electrolysis cells compared to the controls regardless of the post-treatment applied to the biomass digestate. Nonetheless, the methane increase upon voltage addition for the post-treated digestate was higher than that for the non-treated digestate (178% compared to 68%) (FIG. 11, filled markers vs open markers) alluding to the favorability of the post-treatment towards methane production in microbial electrolysis cells. Interestingly, methane production was slightly reduced in the no-voltage control as a result of the post-treatment, suggesting the possible formation of inhibitors during the treatment. These results further provide evidence towards the suitability of the microbial electrolysis cells with other pre or post treatments options already available in the market.

1. A method for producing product gas comprising methane,
the method comprising the steps of providing a biomass,
subjecting the biomass to an anaerobic digestion to produce biogas and a biomass digestate,
separating the biomass digestate into a liquid digestate fraction and a solid digestate fraction,
subjecting the liquid digestate fraction to microbial electrolysis cell processing to produce methane and/or hydrogen gas.

2. The method according to claim 1, wherein the anaerobic digestion comprises a first anaerobic digestion step and a second anaerobic digestion step.

3-7. (canceled)

8. The method according to claim 1, wherein the biomass is continuously fed to a solid-liquid separation arrangement for separating the biomass digestate BD into a liquid digestate fraction and a solid digestate fraction.

9. (canceled)

10. The method according to claim 1, wherein the product gas consists of biogas and optionally hydrogen gas.

11. The method according to claim 1, wherein the product gas further comprises hydrogen gas produced in the microbial electrolysis cell.

12. (canceled)

13. The method according to claim 1, wherein the method comprises collecting product gas comprising methane from the at least one anaerobic digestion reactor and separately collecting methane gas and/or hydrogen gas from the microbial electrolysis cell.

14. The method according to claim 1, wherein the method further comprises a step of subjecting the biomass or a digestate and/or fraction thereof to a cavitation treatment.

15. The method according to claim 1, wherein the method further comprises a step of subjecting the biomass to a cavitation treatment before anaerobic digestion.

16. The method according to claim 15, wherein the cavitation treatment comprises treating the biomass during the anaerobic digestion.

17-18. (canceled)

19. The method according to claim 15, wherein the cavitation treatment comprises ultrasonic cavitation treatment.

20. The method according to claim 1, wherein the method further comprises a step of pretreating, the step of pretreating comprising subjecting the biomass to a pressure below 2 bar and a temperature in the range of 65 to 100 degrees Celsius.

21. The method according to claim 1, wherein the method further comprises posttreating the biomass digestate before the separation step, where the posttreating comprises subjecting the biomass digestate to a temperature above 150 degrees Celsius.

22. (canceled)

23. The method according to claim 1, wherein the microbial electrolysis cell reactor is a one-chamber microbial electrolysis cell.

24. (canceled)

25. The method according to claim 1, wherein the microbial electrolysis cell processing comprising applying a voltage of no more than 1.8.

26. The method according to claim 1, wherein the microbial electrolysis processing is performed in at least one microbial electrolysis cell reactor having a capacity of at least 5 m3.

27. (canceled)

28. The method according to claim 1, wherein the method comprises adding a high energy biomass fraction to the biomass digestate before and/or during said microbial electrolysis cell processing.

29-31. (canceled)

32. The method according to claim 1, wherein the liquid digestate fraction has a dry matter content of no more than 10% by weight of the liquid digestate fraction.

33. (canceled)

34. The method according to claim 1, wherein the solid digestate fraction has a water content of no more than 85% by weight of the solid digestate fraction.

35. The method according to claim 1, wherein the biomass has a dry matter content of at least 5% by weight of the biomass.

36. The method according to claim 1, wherein the biomass comprises farm-based components.

37. The method according to claim 1, wherein the biomass comprises fibrous biomass.

38. (canceled)

39. The method according to claim 1, wherein the biomass comprises one or more selected from the group consisting of straw, deep litter, hay, corn straw, grass, and any combination thereof.

40. (canceled)

41. The method according to claim 1, wherein the biomass comprises a content of animal feces and bedding material of at least 50% by weight of the biomass.

42-47. (canceled)

48. The method according to claim 1, wherein the method further comprises upgrading the biogas by reducing the content of carbon dioxide in the biogas.

49-54. (canceled)
